(19) United States
(12) Patent Application Publication
(10) Pub. No.: US 2005/0215953 A1
(43) Pub. Date: Sep. 29, 2005

(54) Intravenous catheter and I.V. medical line securement dressing and stabilizer for human and veterinary medicine

(76) Inventor: Joel Stephen Rossen, Palm City, FL (US)

Correspondence Address: JOEL S. ROSSEN, 1849 SW CRANE CREEK AVE., PALM CITY, FL, 34990, US

(21) Appl. No.: 11/092,035

(22) Filed: Mar. 28, 2005

Related U.S. Application Data
(63) Continuation of application No. 10/906,630, filed on Feb. 28, 2005.
(60) Provisional application No. 60/556,682, filed on Mar. 27, 2004.

(51) Int. Cl. A61M 5/32

(52) U.S. Cl. 604/180

(57) ABSTRACT

A preemptive bandaging device is presented for the facilitation of intravenous catheter placement and anchoring. The bandage of the present invention includes a frame of bandage base material and a hingedly attached door of a thin bandaging film, a layer of carrier paper to serve as a deployer, and an optional bandage base flap or island for integrating the present invention with certain tube and catheter stabilization devices. The dressing is anchored to the skin so as to frame the I.V. site. The bandage features anchors for I.V. tubing and is placed so as to frame the I.V. procedure site. Once the catheter is placed, deploying the door covers and secures the tubes and the catheter with a thin film of clear bandaging material.

### CROSS-REFERENCE TO RELATED APPLICATIONS

This invention is a further simplification and specialization of the invention disclosed in this inventor's previous provisional application Ser. No. 60/499,118 filed Aug. 29, 2003 and it claims priority to this inventor's provisional patent 60/556,682, filed Mar. 29, 2004 and is a continuation of pending patent application Ser. No. 10/906,630, filed Feb. 28, 2005. The background of the invention remains the same and reference is made to the previous applications.

### FEDERALLY SPONSORED RESEARCH

NA

### SEQUENCE LISTING OR PROGRAM

NA

### BACKGROUND OF THE INVENTION

For a better understanding of the present invention, together with other and further objects thereof, reference is made to the following description, taken in conjunction with the accompanying drawings, wherein like numerals refer to like elements throughout the several views, and its scope will be pointed out in the appended claims.

### FIELD OF THE INVENTION

The present invention relates to dressings to dress and to facilitate stabilization of indwelling vascular catheters. More specifically, this invention relates to the facilitation of one-handed application of such dressings by providing a dressing and methods whereby the dressings can be placed on a procedure site prior to the performance of a medical procedure, the medical procedure can be performed, and the bandaging and catheter stabilizing procedures can be completed virtually simultaneous with the completion of the catheter placement.

### DESCRIPTION OF RELATED ART

Healthcare workers are at risk of serious infections if exposed to pathogens which are commonly present on needles and other sharp devices after such devices are used to break, cut, or puncture the skin of a patient. It is widely recognized that body fluids containing bloodborne pathogens are a serious vector of transmission of infectious diseases. The spread of bloodborne pathogens to clinical practitioners and others by contact with the body fluids of an infected patient is an inherent risk that is routinely taken when conducting procedures involving skin punctures which may expose the provider to blood and other body fluids. Further, certain procedures require that a provider infuse fluids into a patient that may be harmful to the provider. This could include, for example, when providers provide medications to which they themselves may be allergic. Vascular catheterization is one of the procedures that carries with it inherent risks of exposure to bloodborne pathogens. When a catheter is first placed, blood commonly escapes from the system, thereby contaminating the field. The provider is then tasked to secure the catheter, dispose of the cannula, dress the site, secure the I.V. line and avoid undue exposure to contaminated matter.

As such, numerous protocols and medical devices have been developed in order to minimize risks. In particular, catheter anchors, I.V. medical line anchors and stabilizers, safe needle handling, needle disposal practices, needle covers, and needle retractors help to prevent inadvertent needle sticks with contaminated sharps. Some of the developments, such as needle retractors, have brought with them new problems. Some needle retractors, for example, retract with such velocity that the contents of the needle barrels, which may include infectious bio-matter as well as toxic pharmaceuticals, may be aerosolized and even splashed back towards the healthcare provider. Puncture site coverings and wound coverings protect others from contact with post-procedure fluids and exudates. Numerous types of absorbent and adhesive bandages are known in the art that can be applied to a puncture site or vaccination site on a patient. In general, these bandages include an absorbent material that covers the procedure site and an adhesive to keep the absorbent material and procedure site contents in place and to isolated. The bandages protect the patient from microbial contamination of the broken skin while healing and also protect practitioners from the body fluids that can shoot, spray, or seep from the wounds. Although traditional bandages perform these functions, to a certain extent, they do not offer the advantages that accompany rapid deployment nor do they offer needlestick injury protection. Further, in the past, it has not been possible to complete the anchoring of a catheter and the covering a procedure site simultaneously, because the technology simply did not address that possibility.

Certain devices exist in the market now for rapid stabilization of I.V. lines and catheters, but they are intended to be placed on the patient after the catheter has been placed in the vein, opening up the operation to the possibility of losing the vein while trying to stabilize the catheter and tubing.

### SUMMARY OF THE INVENTION

This invention relates to a preemptive patch for dressing and stabilizing intravenous lines and catheters. In both veterinary and human medicine, after placing an I.V. or arterial line or a catheter or a needle into the lumen of a blood vessel, stabilization of the catheter and the I.V. set and safe disposal of contaminated waste becomes an immediate consideration. Taping down of the tubes and stabilization of the catheter can be a clumsy activity, often resulting in the loosening or accidental removal of the catheter or loss of proper catheter placement or extravasation of fluids.

Additionally, in veterinary medicine, the task of stabilizing an indwelling vascular catheter, after it has been placed in a vascular lumen, can be a daunting task requiring securing the catheter to tape and then securing the tape to the animal. During catheterization and once a catheter has been placed, there is no assurance that the patient will not be jumping or running away or biting at it or biting the provider. Rapidly and securely stabilizing the catheter is of primary importance and the temporal window of opportunity to stabilize a catheter may be small.

Additionally, although the invention is designed to function with currently available intravenous catheters and even with luer hub needles, the disclosure also includes embodiments that further include modified catheter hubs and modified catheter adapters that work better with the dressing of the present invention. These modified catheters have what are called grabber extensions on them. Such extensions are typically composed of the same material as the catheter hubs. The extensions are molded into the catheter hubs and parallel the vessel when the device is placed in its lumen. Embodiments are foreseen wherein the extension is flexible or even hinged, such that it could be positioned out of the way or the provider while the device is being placed in the lumen, and then it can be moved into close proximity with the skin after the catheter has been placed.

The hub of a catheter is rather small and the typical hub of a needle is even smaller. As such, a catheter must be placed and positioned such that the hub is inside the boundary of the film of the deployment door. Hence, extending the

An embodiment is also disclosed wherein the grabber extension is a component of a catheter adapter. In an embodiment of this type, the catheter can be placed in the vessel and then, after the placement, plugging the adapter into the female catheter hubs positions the grabber extension parallel to the catheter. The effect of placing and positioning a grabber extension as described is that the dressing film will have more material to grab when the dressing is deployed in addition to the hub.

This application discloses a device designed to overcome the problems associated with securing and dressing an I.V. catheter placement. This application presents a device that is placed on the patient prior to placement of an I.V. catheter. It presents several variations of the device with varying levels of convenience and utility. It addresses the problems, in part, by changing the procedure's bandaging syntax and method, so that the bandaging and stabilization of the catheter and potentially the I.V. lines as well, occur partially prior to the catheterization of the blood vessel and completely within moments after. Although I.V. traditionally refers to intravenous, it will be used in this document to refer to any invasive medical device placement including, but not limited to, subcutaneous, intravenous and intra-arterial placements.

When affixed to skin prior to an I.V. procedure, such a device would decrease the time that would be needed to stabilize the catheter, once the catheter had been placed. It could pre-stabilize the I.V. line using one or more of several means and methods. If fact, it would increase safety and decrease the overall catheterization time, from start to finish, because the bandaging procedure is very rapid and further incorporates the catheter stabilization and the medical tube stabilization, which are two additional procedures.

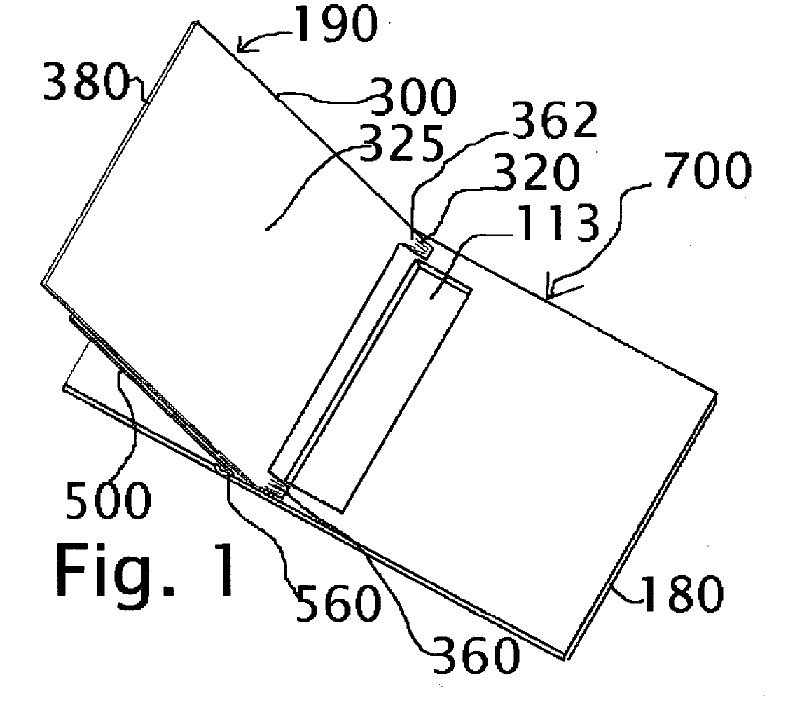

The device can be supplied flat, in a cocked state using a retention extension or it can be supplied flat in an un-cocked stated, using the deployer dual retention leg configuration of FIG. 1. Such a configuration can be cocked after it is placed on the patient by opening the door and folding the legs under. The cocking procedure has been disclosed in this inventor's previous utility application, 10/906630.

At least one embodiment of the present invention has an I.V. extension line affixed to the patch itself. The device can be provided with this extension already attached or the extension can be included, separate from the device, as an accessory to the dressing. As part of the dressing, rather than connected to the I.V. fluid source, this extension facilitates changing the I.V. line without approaching the catheter. The medical supply line could be disconnected from the extension and the extension could be capped off, as needed, to provide patient mobility, such as for a shower. Because the directional orientation of the extension tube will determine or at least suggest the handedness of the provider, it is preferable that the provider place the tube for his or her comfort. Typically, a right-handed provider will place the tube and the door to the left and a left handed provider will place the tube and the door to the right. With the dressing oriented as just described, the hand that places the catheter into the vessel will be on the unobstructed side of the dressing.

Another accessory to the preemptive I.V. stabilization dressing is a hub anchor stabilizer pad or hub anchor stabilizer island, herein, the island, where a hub anchor is a device intended to secure the hub of an I.V. line or that of a catheter. Numerous devices have been described in the literature for stabilizing tubes and tubing hubs after the catheter has been inserted. These devices or catheter anchors, must be placed after the catheter is placed to make sure that they are in the correct location. An optional member of the present invention is an island that serves as a pad for such an anchor. An island, composed of a section of flexible bandage base with a small tongue of bandage base, could be included with the dressing. The bandage base material, with a protective carrier paper layer on the bottom, could be adhered to the bottom cross-member of the frame by the tongue. Then a catheter anchoring device, such as disclosed by 6,837,875 Bierman, Jan. 4, 2005, could be anchored to the dressing on the island, prior to placing the catheter. While the Bierman '875 device was developed for use after the catheter was already in place, anchoring a flexible bandage base material to a cross-member of the present invention would provide a movable catheter anchor base, such that, after placing the preemptive dressing, a catheter anchor could be placed on the island and could still be moved so the its receptacle could be easily positioned to coincide with the tube hub. In particular, that of Bierman '875 being somewhat positionally forgiving would be a logical choice for incorporation into, or for use with a device of this nature. Having been somewhat stabilized prior to the insertion of the catheter, fumbling, to place the catheter anchor while protecting the catheter placement, is minimized. The catheter anchor can be anchored to the island prior to the placement of the catheter in the vessel. Because the island can be fitted with an optional tongue, a catheter anchor can be affixed to it and the island can be anchored to the bandage base or to the skin by adhesive on the bottom surface of the tongue, yet, it can still be easily moved small distances, sufficient to place a medical tube into the anchor, until the removal of the carrier paper on the bottom of the body of the island, at which time it is anchored to the skin and stabilized. Many catheter anchoring systems and devices are currently available in the marketplace and others, not yet available, are covered by patents and pending patents. It is obvious to any person skilled in the art that substitution of one catheter anchoring device for another would still provide a device within the scope of the invention. Obvious changes in the relative sizes and shapes of the catheter islands and changes in the catheter anchoring device with which it might be paired, could produce a myriad of similar devices that all would fall within the scope of the invention.

Numerous I.V. medical tube sets and I.V. set extension devices exist in the medical world. These devices are fitted with accessories chosen from a myriad of adapters, including, but not limited to, stopcocks, y-sites, caps, bifurcations, rate controllers, and slide clamps. The novelty of the present invention is further enhanced because elements are herein disclosed for affixing such I.V. sets and I.V. extension devices to the invention. In this way, the I.V. tubing can be anchored or stabilized, prior to starting the catheterization. Configurations are disclosed herein such that, although anchored, the male end of the tube that inserts into the female hub receiver of the catheter would remain mobile, within a limited range and a I.V. tube anchor can be placed, preemptively, on an island, where the anchor could secure the tube male hub, prior to inserting the catheter in the lumen of a blood vessel. In this way, when the catheter is placed in the vessel and it is time to connect the I.V. line to the catheter, the I.V. catheter adapter is in close proximity to the catheter hub and only a small movement is needed to place it and complete the connection. Embodiments of the invention are foreseen wherein the present invention might be combined with any or all of the accessories named in this paragraph.

When using any embodiment that includes an integrated or a separate extension tube section, tube stabilization could be incorporated into the tube extension design. The tube stabilizer could be as simple as a binary contact adhesive on the tube that is non-adhesive until placed into contact with its counterpart on the bandage base, or an adhesive patch surface that is exposed when a carrier paper on the frame cross-member is removed to allow the tube or the adhesive of the two adhesive coated members to come into contact with one another. Some binary adhesives do not require being covered by a carrier paper protector. A more aggressive adhesive could be used, on the cross-member only, to grab an uncoated tube when the tube is pressed against the cross-member.

Inclusion of a tube extension with the dressing opens many avenues of tube anchoring. For example, a sliding tube anchor could be included with the device such that; a.) the tube anchor could be a molded plastic ring with a flat base and the tubing could be threaded through such a ring before the molded hubs were placed on the ends of the tube so that the anchor would easily slide on the tube and could be placed on the island or on the cross-member and then, once the catheter has been inserted in the lumen of the vessel, the tube would slide through the ring so its male connector hub could enter the female catheter hub and close the fluid circuit.

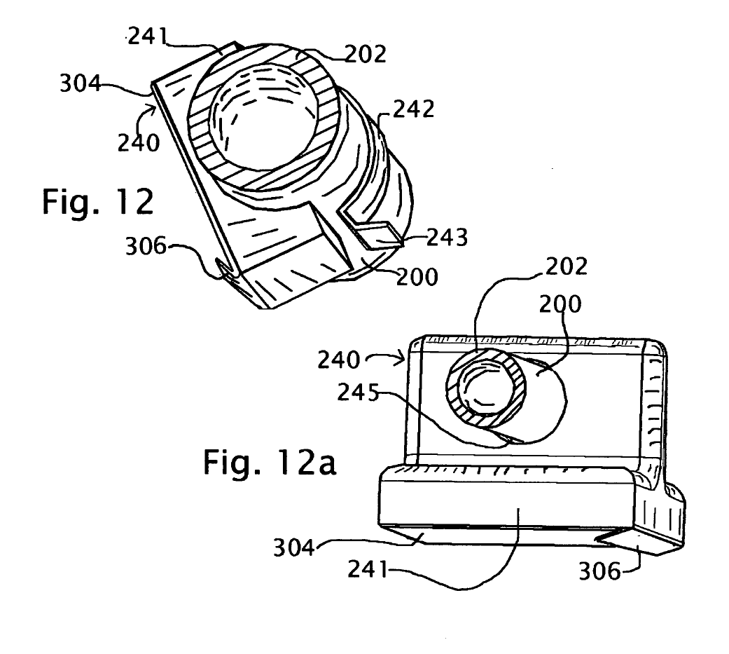

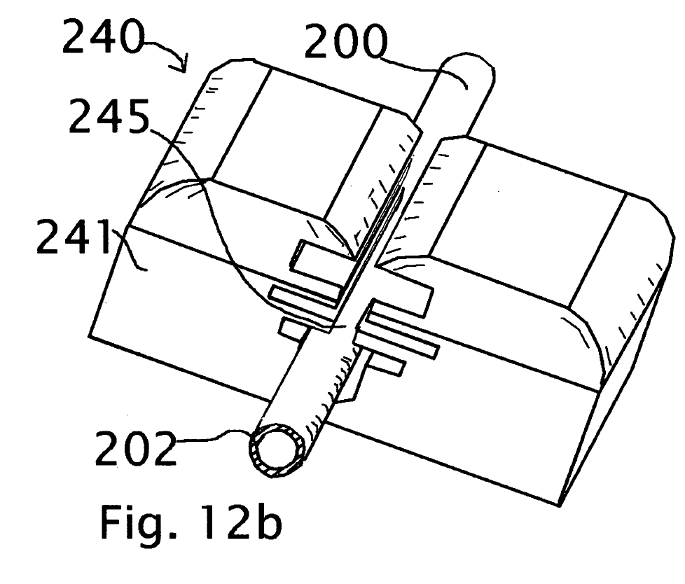

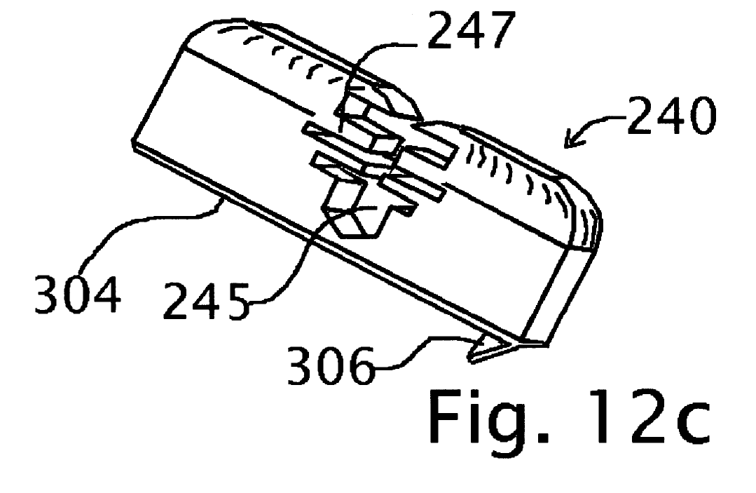

Tube anchor options include bi-digital ring of plastic, like a thumb and forefinger coming together or a child's adjustable toy ring or a plastic clamp, similar to the bi-digital ring, each of which could exert variable pressure against the tube, such that the tube could be moved through the bi-digital ring as needed and the ring would grasp the tube with sufficient force to anchor the ring to the tube wherever the tube was released FIG. 12. This way, the tube could be preemptively anchored to the dressing, yet would still be positionable. A tube anchor, such as that of FIG. 12a with tunnel to trap the tube, so that the tube was limited to moving in only 2 dimensions would also work. Both of these anchors are disclosed in greater detail below. An anchor similar to the embodiment of FIG. 12a is foreseen with a tunnel with sufficient diameter to pass a luer connector, therefore obviating the need to place the tube into the anchor until just before the procedure.

Additionally, a device of this nature is in alignment with the intent of the current needlestick injury prevention guidelines, because it minimizes the likelihood that the catheter would slip from the vein, exposing healthcare providers or third parties to the inevitable spillage of body fluids and it permits the provider to immediately focus on the safe disposal of the cannula.

This invention has unique properties which are extensions and variations and specializations of those disclosed in provisional application Ser. No. 60/499,118, filed Aug. 29, 2003 and shows a method of simplification of the invention of Ser. No. 60/499,188. This device shares the property of being used preemptively, that is, before the skin is penetrated and may use a window through which to access the vein. It has an adhesively hinged door which is closed to complete the bandaging process immediately after catheterization. This device is unique in that it may incorporate either an I.V. tube extension or an adhesive mount for the I.V. tube into its design and that it has a film, typically clear, which is attached to a door than can be closed over the catheter and tube end, to rapidly secure the intravenous infusion device(s) in place. The film may be opaque but in the preferred embodiment, the film is clear and is adhesively coated on the surface that covers and anchors the catheter. Disclosures prior to 60/499,188 did not speak to catheter nor to I.V. line stabilization.

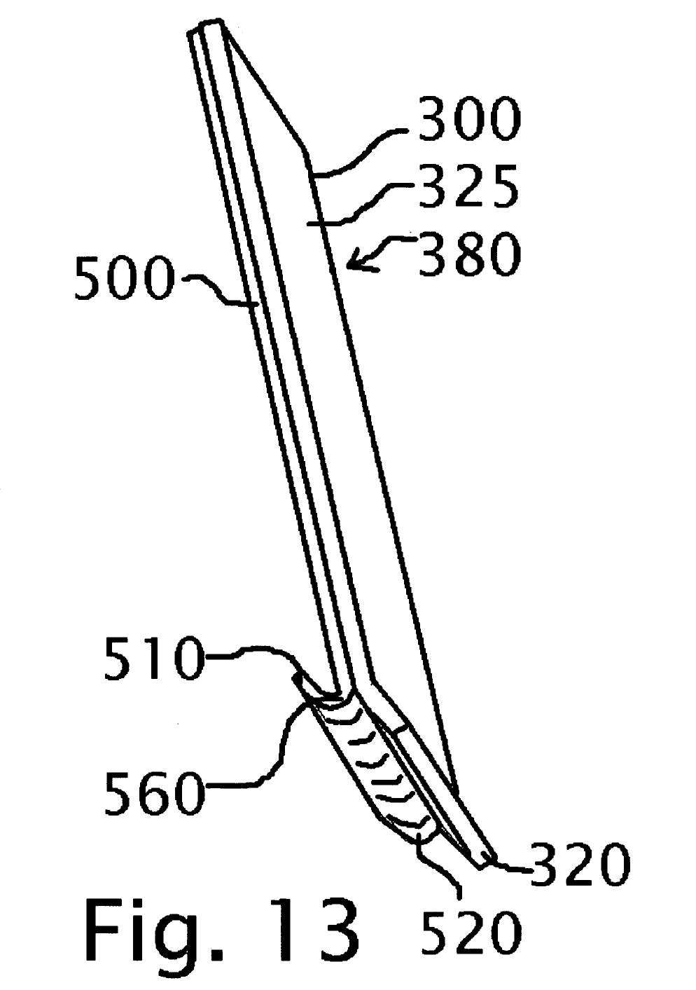

In it simplest form, embodied in FIG. 13, the dressing of the present invention may comprise nothing more than a deployment door made of a piece of tape affixed to a piece of carrier paper. Exposing one edge of the tape exposes a strip of adhesive on one edge of the tape. That strip can be adhered to skin or to a bandage base. Then the portion of the tape that is still affixed to carrier paper can be folded at the edge of the carrier paper that transverses the adhesive layer of the film. The fold, in essence, hingedly affixes the deployment door, which is the carrier paper and all film affixed to it, to the skin. The deployment door can be folded about the hinge and the portion or the carrier paper that is not affixed directly to film becomes a retention extension. When the dressing tries to unfold, the retention extension will contact the skin before the dressing unfold more than about 90 degrees, resulting in a dressing that is cocked on the skin. Various means of preventing the folded dressing from unfolding are presented in this application.

Most of the embodiments described herein also comprise a bandage base layer in addition to the deployment door of the previous paragraph. The preferred embodiment of the present invention includes a deployment door and a bandage base, made of traditional bandaging materials, such as, but not limited to plastic, cloth, cotton, polyurethane, or foam, and adhesive coatings. The base may be a simple piece of foam or tape, coated with adhesive on one side, or it may further include an optional absorbent pad. Such a pad could be located many places on the tape, including part on/part off, depending upon the intended use of the device.

This bandaging device may further comprise medical tubing. The bandage base has a top surface where an I.V. tube can be quickly anchored during the catheterization process. It may have an I.V. tube extension affixed to it, one end with a female receptacle for receiving the male hub end of an I.V. tube set and the other end with a male connector to fit directly into the catheter, typically a luer type connector. Typically, the end of the tube that is plugged into the catheter hangs loose until after the catheter is placed in the blood vessel. Then the tube's catheter adapter is plugged into the female hub of the catheter. Then a protective carrier paper cover on the adhesive anchor is removed so the end of the tube extension can be adhered to the adhesive on the top of the bandage base; and hence, the tube is rapidly stabilized.

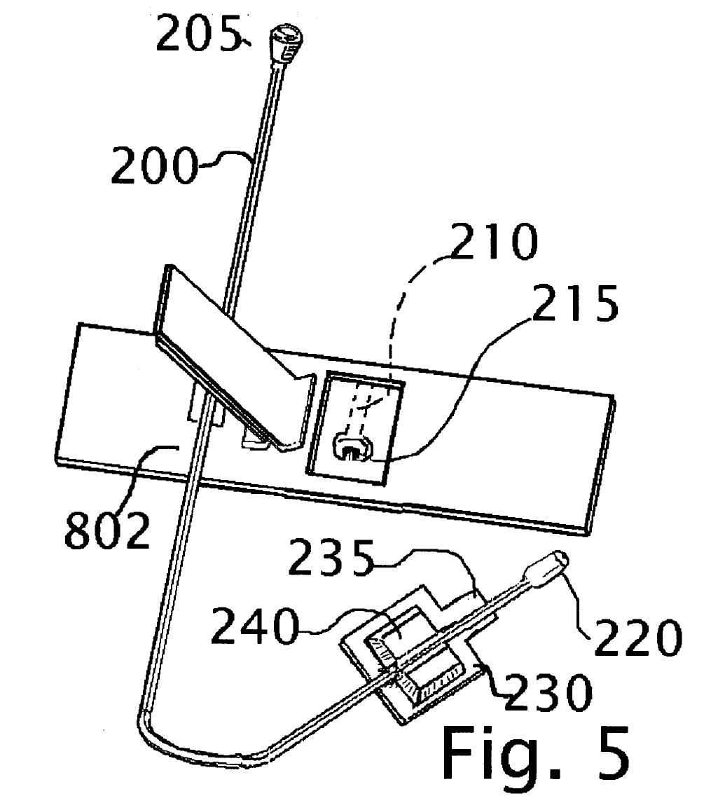

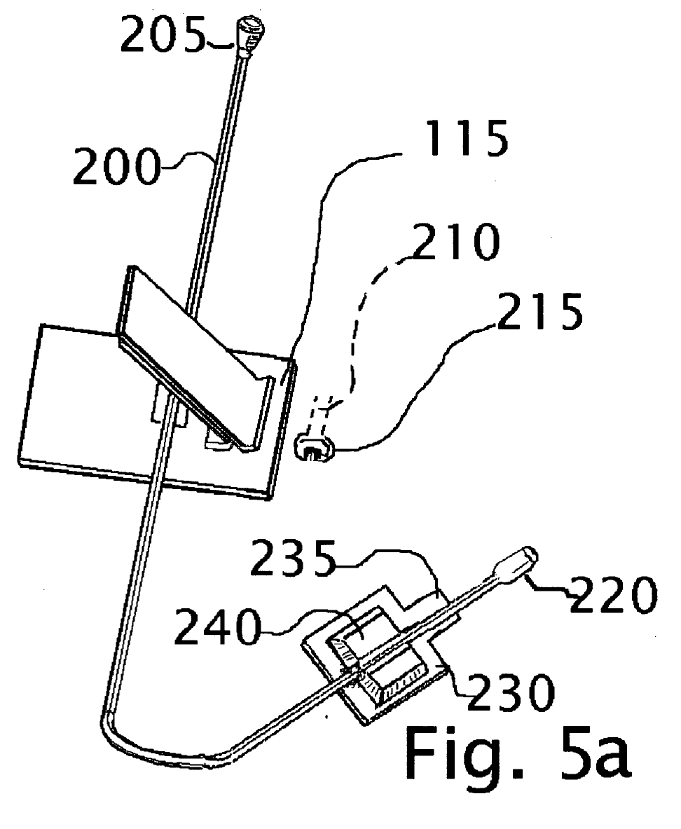

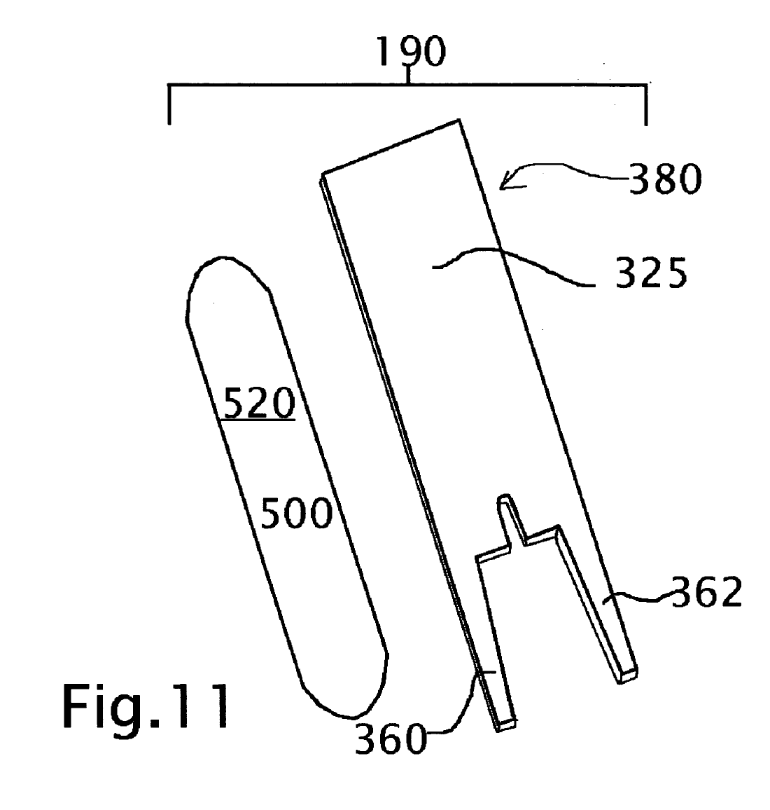

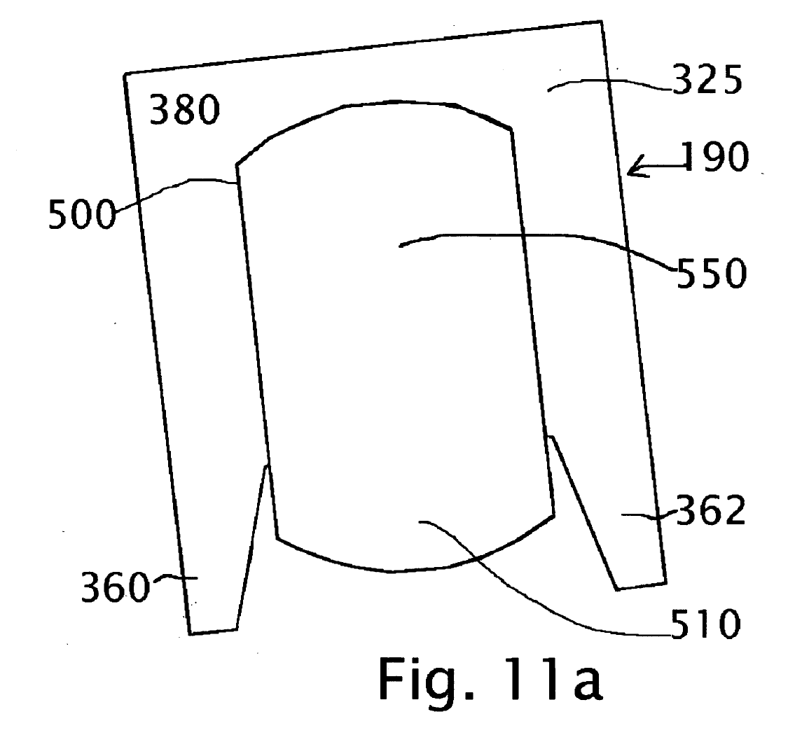

The device is adhesively affixed to skin prior to an injection or medical procedure. Once pre-placed, all components of the dressing remain clear of the operative field until it is needed. The section of the bandage that contacts the skin may have a portion removed to define an aperture or it may be designed in such a way that it can be placed close to the procedure site so as to infer the procedure's intended location from the positioning of specific components of the bandage. The less expansive embodiment, the embodiment with neither a frame nor an aperture, such as those embodiments disclosed herein shown in FIGS. 5a and 11 and 11a are less expensive to manufacture.

The present invention further includes methods of applying dressings, including the embodiments disclosed herein, to human or animal tissue, prior to the inception of an invasive medical procedure, such as an intravenous catheterization. The present invention includes means and methods of retaining said bandage in an un-deployed or cocked state and preventing it from entering the operative field prematurely.

It is widely recognized that body fluids containing bloodborne pathogens are a dangerous and serious vector of transmission of infectious diseases. The spread of bloodborne pathogens to clinical practitioners and others by contact with the body fluids of an infected patient is an inherent risk that is routinely taken when conducting procedures involving skin punctures and releasing blood and other body fluids. Further, certain procedures require injection of infusion with materials that may in themselves be potentially infectious or dangerous to the provider. Some medical practitioners are, themselves, allergic or sensitive to certain of the medications or drugs that they provide to patients. Penicillin is a good example. Because this device rapidly anchors catheters and tubing, opportunities for the escape of body fluids from the invasive medical intervention site are minimized. Prevention of exposure of these practitioners to sharps-borne and blood-borne materials that may cause them harm is an important benefit of this invention.

It would be advantageous to have a procedure site dressing available to clinicians that overcame the above-cited disadvantages. In particular, it would be desirable to have available a dressing that permitted the clinician to apply the dressing before performing an invasive procedure so that the contaminated sharps could be discarded without the clinician have to simultaneously bandage the site. Additionally, it would be advantageous to have a device that stabilized a catheter placement without requiring further handling or taping of the catheter or tubing.

### DESCRIPTION OF The INVENTION

The invention is essentially a device that stabilizes an intravenous line during and after the process of intravenous catheterization. Embodiments disclosed herein include tubes that extend the reach of standard I.V. tubes. The value of this is that the extension tube can be anchored to a piece of bandaging material which is adhered to the skin prior to the introduction of a catheter. An embodiment of the device, disclosed herein, has an aperture through the base of the patch that clearly defines the procedure site where the catheter will be introduced. In another embodiment, rather than an aperture, the portion of the bandage that actually frames the skin site is missing and the procedure site is inferred from the position of the hinge of the preemptive bandage.

To use the device with an I.V. line extension: 1. The device is placed on the skin with the I.V. site visible through the aperture. 2. The I.V. line is attached or plugged into to the extension. 3. The extension is filled with fluid from the drip bottle, that is, the air is purged and flow is halted. 4. The catheter is placed in the vein and then the male plug of the extension is plugged into the catheter. Until the extension is plugged into the catheter, the male catheter end of the extension remains free or partially controlled by a hub anchor, to make it easy to control the tube and to plug it into the catheter. 5. Positioned between the bandage base and the catheter, there may be a piece of adhesive, covered by release paper. When the release paper is removed, the adhesive is exposed and simply pressing the extension tube against the adhesive anchors it. Depending on the technique being used, the extension can be plugged into the catheter before or after the catheter is placed in the vein. Optionally, the extension tube can be pre-loaded with certain fluids or medications.

The basic invention is a hinged or cockable hinged door of dressing material. The door has two or three layers. One or two of the layers are release paper and the other is an adhesive coated bandaging material, such as polyurethane, typically clear. When the door is cocked, pulling the tabs or retention extension on the release paper rotates the adhesive into contact with the catheter and the tube extension and instantly anchors them both in place. Techniques for using the invention will vary by provider and the needs dictated by the procedure for which it is being used. Sometimes just the catheter will be secured by the door and sometimes both the catheter and the I.V. tube will be secured by the film.

Alternatively, if a layer of release material is also attached to the non-adhesive side or back of the polyurethane, the inner release paper, that affixed to the adhesive, is be removed first to expose the full layer of adhesive. Then the door can be rotated to bring the adhesive flat against the tube and the base frame and the skin, all at once. Pressing on the non-adhesive surface of the door to push the adhesive of the film layer against the catheter and tube will anchor the catheter and tube and door in place. Finally, the top piece of release paper can be removed to expose the clear polyurethane. This then permits the catheter and tube and skin and vein to become viewable through the clear bandage window.

A variation of this device is designed especially for use in veterinary and pediatric medicine. In this embodiment, the base, beyond the aperture which exposes the vein, is much wider. In fact, it is wide enough that the adhesive can wrap all the way around a limb until the two ends meet. This addresses the problems that occur when one tries to tape an I.V. tube and catheter to the leg of an animal or a small person. The actual size of this device will vary greatly, depending upon the size of the animal that it will be used on. It could range from a total size that would be small enough for a kitten to one large enough for a horse or even an elephant.

Some embodiments of the invention do not have the I.V. tube extension tubes included. In these embodiments, the location where the I.V. tube is to be placed, where the extension tube was on the previous embodiment, may have an adhesive strip on it. The adhesive is covered and protected by a protective piece of release paper. When the device is used, it is first placed over the vein, and then the I.V. tube is set up. The release paper is removed from the adhesive strip and the adhesive is exposed. Then the tube is anchored to the adhesive strip, the catheter is placed in the vein and linked to the I.V. tube. Then removing the protective paper exposes the adhesive just below the catheter. Then the tube and the catheter are anchored to the base. The deployer is used to deploy the dressing, to close the door as with the others embodiments.

By providing a bandage that is placed over or near the procedure site prior to the invasive part of the procedure, the bandage itself becomes an integral part of the catheter placement procedure and can be used to complete the bandaging process within moments of, or virtually synchronous with, the completion of the procedure. This improved syntax frees the provider to dispose of the contaminated waste materials immediately, rather than after to maintain possession of contaminated materials with one hand while simultaneously performing a difficult bandaging procedure on the site with the other hand. This dressing rapidly, firmly, and safely stabilizes the I.V. tubes and catheters.

It has now been discovered that certain methods of folding, taping, placing, and the applying the bandages disclosed in this application will greatly reduce the time it takes to complete the bandaging of a procedure site after the invasive part of the procedure has been performed. Additionally, because the bandage is applied before the procedure has been performed, not after performing a puncture, when using this invention, healthcare providers need not choose between disposing of a contaminated sharp, thus protecting themselves, and bandaging the site while maintaining possession of the contaminated sharp, thus protecting their patient.

The invention is a dressing with a bandage base frame and a door, also called a deployment door, typically of a thin, transparent, adhesively coated bandaging membrane, such as a polyurethane tape or film weakly adhered to a carrier paper layer. The door is hingedly affixed to the bandage base by an adhesively coated edge of the film. The edge that affixes the film to the bandage base is called the anchor-flap. Except for the small amount to adhesive film that affixes the door to the frame, the adhesive surface of the door is up, and the door's adhesive surface is covered with release paper over its entire adhesive surface. The bandaging film used is any one of the many bandaging films available in sheet and roll form, wherein a release paper layer separates the contiguous sheets or layers of film and prevents them from adhering to one another. The film is hingedly affixed to the frame of the bandage base as a component of a deployment door. The film can be many sizes. It may be smaller than a strip of bandaging tape or as wide or wider than the bandage base of the dressing. Films of this type are supplied by manufacturers such as Avery Label and 3M and the film is weakly adhered, as delivered, to a carrier paper covering. The film, with its carrier paper layer, which will function as a deployer, together, comprise the deployment door. To affix the deployment door to the bandage base, some carrier paper is separated from a strip of film at the long edge of the door. The width of the strip must be sufficient to form the base of a hinge. Typically, the length of the door is about the same as that of the frame, although it can be smaller or larger than the bandage base.

For a right handed embodiment, the full carrier paper remains intact and the film that has been separated from the paper film is folded about 180 degrees along its length and the adhesive of the strip is adhered directly to the frame, such that the door is open to the left and the door is hingedly adhered to the frame by the now folded under and bent into a hinge, strip of bandage base film. The carrier paper is a deployer. The separated portion of the carrier paper extends beyond the hinge and serves as a retention extension to prevent the door from entering, obstructing, or obscuring the procedure's operative field, both physically and visually. The retention extension also acts as a pull-tab that will be used to deploy the device.

An embodiment of said dressing is foreseen with a plurality of apertures whereby said apertures are labeled or numbered and are placed on a patient for the purpose of allergy testing. Said embodiment is affixed to skin over an area of the body where a series of sensitivity tests are to be performed. A separate antigen could be injected into or dropped onto the skin in each of the apertures and then, when the dressing is deployed, all of the sensitivity testing sites are simultaneously covered with the transparent film. Such an embodiment could have the adhesive of said film over the entire adhesive coated side of the film or it could have windows devoid of adhesive over each of the apertures or, it could have only a frame or adhesive around the outside of the film's adhesive surface to seal the sites, yet not affect the optical quality of the film and not expose said patient to an adhesive that might change the results of said sensitivity testing.

The embodiments intended for veterinary use vary from their human counterparts primarily by size and shape. Adhering a dressing to human skin is a relatively straight forward process, whereas, indeterminate quantities, qualities, and varieties of hair often make adhering a dressing directly to animal skin nearly impossible. Shaving the patient may be the only way to address the situation. The dressing of the present invention addresses this problem by being available in many sizes, suitable for wrapping all the way around a limb that has been chosen as a catheter insertion site. While the dressing would adhere best to a fully shaved limb, that is not always practical. The size of dressing chosen is intended to be adequate to wrap around the limb and adhere to itself. The size of the window in the frame of a veterinary dressing of the present invention will tend to be smaller, relative to the side of the entire dressing. The size of the door will be smaller than the human counterpart, relative to the size of the bandage, but will be the similar, relative to the size of the procedure window.

The embodiments provided herein as examples are for illustrative purposes and are non-limiting. An embodiment is foreseen wherein the bandaging film is far smaller than the bandage base and when the device is deployed, only a strip or a plurality of strips crosses the operative field, such that the tube and the catheter are bound separately or only the tube or only the catheter is bound by the film of the door.

### NOVELTY OF THE INVENTION

The present invention is a preemptive dressing for stabilization of I.V. catheters. In contrast to all other known methods of catheter stabilization, the device is placed over or near the catheterization site prior to placing the catheter into the lumen of a blood vessel.

Prior art that shows devices meant to hold catheters and/or I.V. tubes in place speak to devices that are intended to be placed on the skin after the catheter has been placed. This is inevitably a clumsy operation because one must maintain the placement of the catheter or needle and manage the I.V. tube and connecting hubs, while simultaneously anchoring the devices with tapes of other devices that are to anchor them.

In addition to catheter stabilization, the device also can preemptively anchor I.V. tubes. Previously existing device are designed to anchor only one or the other, not to anchor and dress both. The dressing of the present invention is designed so that an I.V. tube or an I.V. tube extension can be anchored to the body of the device before the puncture is made. With the tube pre-anchored, only the catheter and the male catheter adapter end of the I.V. tube need to be stabilized after the catheter is placed.

In prior art, catheters are often stabilized by wrapping a piece of tape around the hub. Handling tape with gloves on is a delicate and often disappointing endeavor. The invention eliminates tape handling for catheter securement. The initial placement of the dressing coincides with anchoring of the tubing and precedes the placement of the catheter. Placement of the catheter is followed, within fractions of a second, almost immediately, by stable anchoring of the catheter without having to fuss with tapes or hub anchors. When the film door is closed over the catheterization site, the bandaging film of the door adheres to the catheter and to the skin or to the bandage base, which is adhered to the skin.

Simply pulling the deployer across the catheterization site, exposes the adhesive of the bandaging film. Closing the door completes the process of stabilizing the I.V. catheter and I.V. tubes. In an embodiment with a three-layer deployer, removing the top deployer of the cocked dressing exposes the adhesive layer of the bandaging film. Then, rotating the door to bring the film into contact with the catheter, instantly stabilizes the catheter, and peeling off the stiffening layer deployer completes the procedure.

In its simplest embodiments, the dressing is a deployment door, which is a layer of carrier paper, called a deployer, affixed to a layer of bandaging film as seen in FIG. 13. Although thin, transparent, medical films are the material of choice for the disclosed embodiments, those skilled in the art would acknowledge that any bandage base material including plastic, cloths, polyurethanes, and even metallic films could be substituted and still be well within the scope of the invention. The film is affixed to the deployer such that, when the deployer is pulled across the field of the procedure, the film peels off, adhesive side towards the procedure, where it adheres to the catheter and anchors the catheter in place, simultaneously stabilizing, dressing, and sealing the procedure site.

In the present invention, a deployment door or a dressing comprising a deployment door is placed on the patient before a catheter is placed in the vessel. Such a deployment door may be anchored to a bandage base, or directly to skin, at many locations near the proposed procedure site and not diverge in any way from the scope of the invention. Such a pre-placement of a preemptive device for the securement of an indwelling catheter is previously unknown.

Additionally, existing catheters are typically like an iceberg in that most of the catheter is unseen once the catheter is placed in a blood vessel. As such, the small portion of the placed catheter that resides outside of the skin is all that the provider has to anchor to tapes or other catheter securement devices to prevent the catheter from moving. A catheter hub grabber extension that can extend from the catheter hub or from a catheter hub adapter is disclosed in this application. This grabber extension that extends the portion of the catheter hub that is external to the skin and makes grabbing and anchoring the catheter an easier procedure because the grabber extension can be easily affixed to the door of the dressing or to a adhesive film to stabilize the indwelling catheter. Such an extension is not limited to I.V. catheters and can also be added to other catheters such as a Foley catheter.

Definition List 1

| Term | Definition |
|---|---|
| Absorbent pad | A piece of absorbent material, such as, but not limited to, cotton |
| | or a synthetic absorbent, such as a thin sheet of hydrophilic |
| | absorbent polyurethane foam. |
| Bandage base | Any flat tape material used in the medical industry to make |
| 180 | adhesive bandages. The bandage base material is coated on |
| | one side with an adhesive that has the necessary adhesive |
| | characteristics to adhere the bandage base to skin. |
| Carrier paper | Any flat material used in the medical industry wherein the |
| or Release | outermost layer of at least one outer surface is comprised of a |
| paper 300 | substance that will resist bonding, other than weakly, to |
| | adhesives, and will protect the integrity of the adhesives. |
| | Carrier paper may be coated paper, coated polyurethane, coated |
| | polyethylene, coated polyester, or any flat material such that at |
| | least one outer surface of the material will resist bonding to adhesives. |
| Catheter 210 | Any medical device placed into the lumen of a blood vessel, |
| | generally for the purpose of infusing a liquid into the vessel |
| | through a concentric device lumen. |
| Cocked | The state of being locked in a predetermined position, yet |
| | having the potential to move into another position when the |
| | element or urge that is preventing it from moving towards its |
| | potential is removed. |
| cocked | A dressing that has been distorted from its flat shape into a |
| dressing | particular stable shape or configuration in which it remains until |
| | and unless acted upon by an urge means. Typically, a cocked |
| | dressing is one that has been folded about a hinge and has been |
| | locked in the folded configuration by at least one element of the |
| | dressing adhering to or being unable to move past skin or |
| | another element of the dressing. |
| Contiguous | Abutting: having a common boundary or edge; touching. |
| To deploy | To purposefully unfold the cocked dressing and bring it to rest |
| | in a predetermined location. |
| Deployer 380 | A device or a member of a bandage that can be used to move a |
| | bandage from one positional configuration to another. Typically, |
| | a deployer protects the adhesive surface of a flip-door, allowing |
| | the flip-door to be handled by a provider. Some element of a |
| | deployer can be grasped and pulled to purposefully unfold a |
| | cocked bandage and bring it to rest in a predetermined location. |
| Deployment | A flip-door and a deployer adhered together. Both parts move |
| door 190 195 | as one until the deployer is removed from the flip-door. |
| Film 500 | Any thin flat material that could be used in the construction of |
| | a medical dressing. |
| Flip-door 550 | A flip-door is the entire dressing, that is bandage base or |
| | bandage base and absorbent pad, except for the anchor-flap. |
| | That is, on the bandaging element that comprises a hinge, the |
| | anchor-flap ends at the hinge and the remainder of that element |
| | is the flip-door, because, that is the portion of the dressing |
| | that flips when it is cocked and flips when it is deployed. |
| Grabber | A protuberance on a catheter hub or on a catheter adapter hub |
| extension 220, | that extends the length of the portion of catheter material, of a |
| 225 | placed catheter, that is external to the skin. Such a grabber |
| | extension typically parallels the subcutaneous portion of the |
| | catheter and is easily grabbed by an adhesively coated |
| | bandaging film to secure the catheter to skin or to a dressing or |
| | both. |
| Hinge 160 | The line in the bandage base or a line in the bandaging film |
| | base, where the dressing is folded when it is cocked. When an |
| | absorbent pad is present, as in the preferred embodiment, the |
| | hinge is generally a line at the intersection of the anchor-flap |
| | and the absorbent pad, although, atypically, a dressing might |
| | have its hinge located under the absorbent pad. |
| I.V. catheter | Any connector on the end of an I.V. line that has the purpose |
| adapter | connecting the line to an intravenous catheter. These are |
| | generally male luer connectors, although proprietary adapters |
| | could fall within the definition. |
| Invasive | A bandage base, generally with an absorbent pad somewhat |
| medical | centrally located on the adhesive surface of the bandage base, |
| procedure site | the proposed purpose of which is to adhere to skin to protect |
| dressing | wounds created during the course of a medical procedure, such |
| | as an injection, from pathogenic invasion, and to prevent body |
| | fluids from migrating beyond the immediate wound site. |
| Catheter | The most likely sharp to be used in conjunction with the present |
| | invention. Typically, a catheter is a medical device with a needle |
| | inside sheath, the two having concentric lumens. When the word |
| | catheter is used in this application, any medical device that |
| | might be used in the procedure, other than specifically a |
| | catheter, is explicitly implied. As such, any catheterization |
| | procedure or procedure cited in this application may also be |
| | read to include any invasive medical procedure, including, but |
| | not limited to, an injection, a surgery, or a debridement, that |
| | might also be performed at the site. |
| Provider | A person who administers the invasive medical procedure that |
| | will be bandaged using the dressing of the present invention. |
| Preemptive | A bandage that is used when a wound is foreseen on presently |
| bandage | unbroken skin. Typically the wound is the result of a skin |
| | puncture, such as from an injection or a catheterization |
| | procedure, or from an incision, as would occur during a surgical |
| | procedure. Prior to the invasive part of such a procedure, such |
| | a bandage is placed on, over, or near the potential wound so |
| | that the dressing can cover the wound as rapidly, often almost |
| | immediately, after the wound occurs. |
| Procedure | Any medical procedure, including any invasive medical |
| | procedure. |
| Retention | A section or piece of a deployer which is generally a sufficient |
| extension 320 | amount of carrier paper material, in addition to the portion of |
| 321 | the deployer that is adhered to a flip-door of a dressing, to |
| | extend the physical boundary of the deployer beyond the hinge |
| | of the flip-door. |
| retention leg | A type of retention extension. Any retention extension that is |
| 360 362 | smaller than the full width of the deployer is a retention leg. |
| Retainer | A means whereby an object is retained. In this application, the |
| | disclosed tube anchors are retainers. |
| Slug 350 | A section of carrier paper that covers the anchor-flap of a |
| | dressing, which, upon removal, permits the dressing to be |
| | affixed to skin, in a configuration such that the dressing can be |
| | subsequently cocked. |

### DRAWING REFERENCE NUMERALS LIST

110 Adhesive surface of bandage base
113 Aperture
114 Flap 1
115 Flap 2
117 Adhesive clasp
118 Non-adhesively coated back side of bandage base or back of bandage base
180 Bandage base
190 2-layer deployment door
195 3-layer deployment door
200 I.V. line or medical tubing extension
202 I.V. line cross section
205 I.V. line or I.V. line extensions medical tubing receiver hub or female luer hub.
210 Catheter
212 Catheter hub extension
215 Catheter female hub
220 Male end of catheter adapter
225 Catheter adapter extension
230 Island
235 Island tongue
240 I.V. line or medical tube anchor
241 IV line or medical tubing anchor base or body
242 Medical tubing expansion retention anchor clamp
243 Medical tubing anchor clamp handle
245 Tube anchor tunnel
247 Flexible fingers
250 Absorbent pad
300 Carrier paper or Release paper
302 Carrier paper 2 or stiffening layer
304 Tube anchor carrier paper layer
306 Tube anchor carrier paper layer pull-tab
320 Retention extension
321 stiffening layer retention extension
325 Deployer body
360 Left leg or left retention leg
362 Right leg or right retention leg
380 Deployer
400 Skin surface
410 Blood vessel
450 extremity or limb
500 Film layer
510 film anchor-flap
520 Adhesive surface of film
530 Back, non-adhesively coated, side of film layer
550 flip-door
560 Hinge
700 The invention or the dressing or the invasive medical procedure site dressing, including at least a film layer, and a deployer.
800 Adhesive coated section
802 Adhesive coated section 2.

### BRIEF EXPLANATION OF THE FIGURES

FIG. 1 is an oblique view of the present invention.

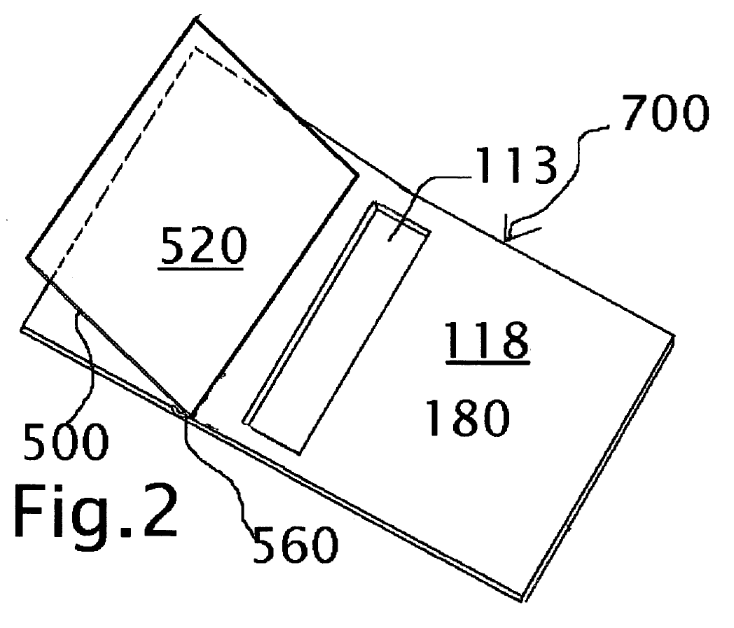

FIG. 2. is the invention of FIG. 1, with the carrier paper layer removed.

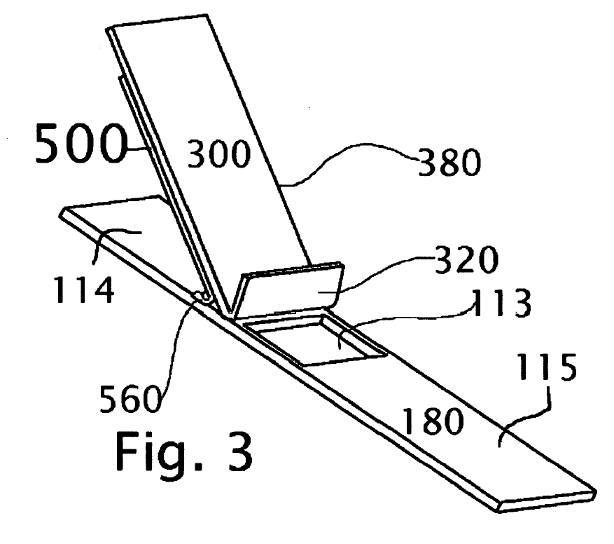

FIG. 3 is an oblique view of the present invention.

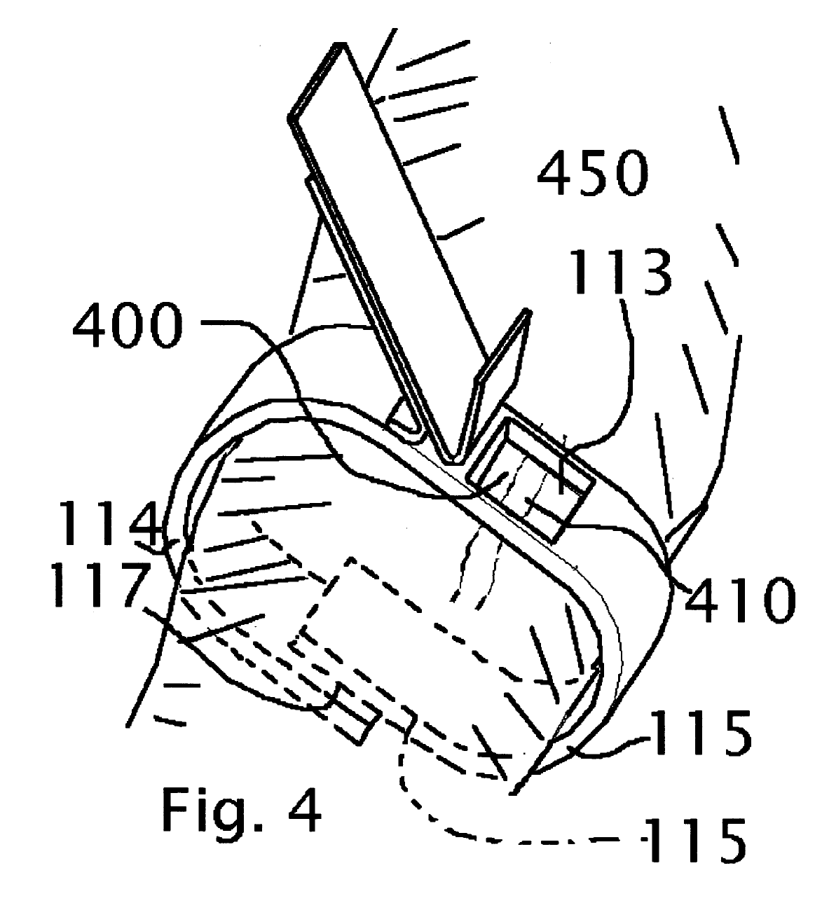

FIG. 4 shows the invention wrapped around a limb and affixed to said limb.

FIG. 5 is a top view of the invention with a medical tube extension, an island, and a medical tube anchor.

FIG. 5a is exactly like FIG. 5, except that one of the bandage base flaps is very short.

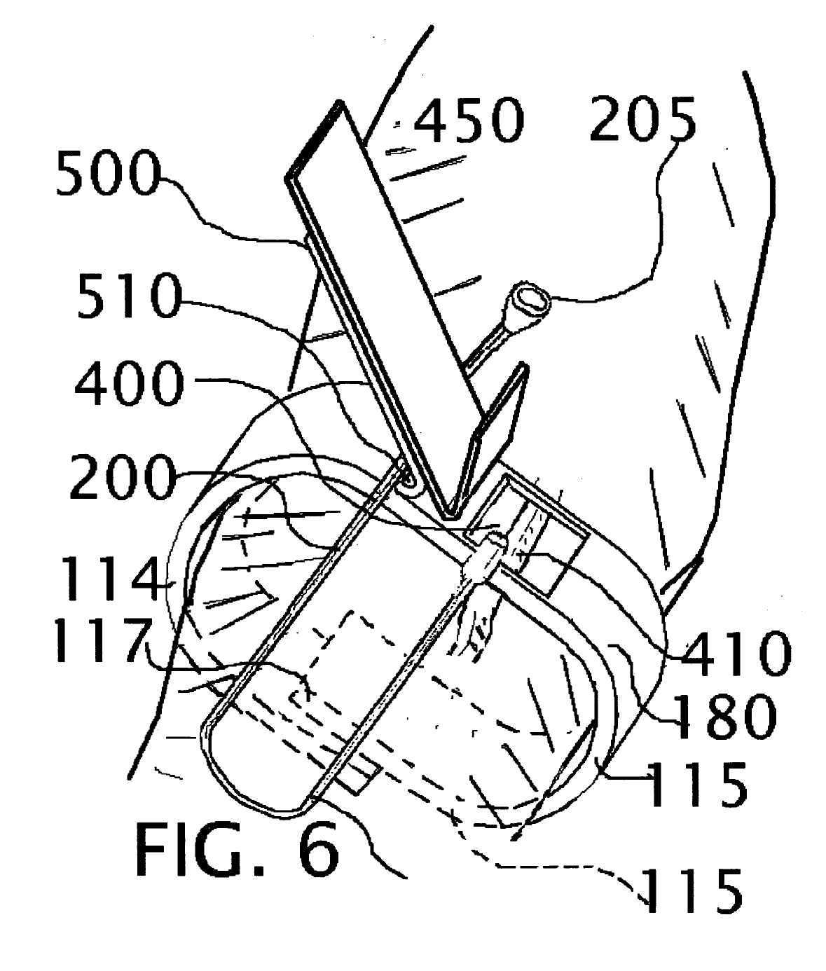

FIG. 6 is the same as FIG. 4, except a medical tube extension has been added.

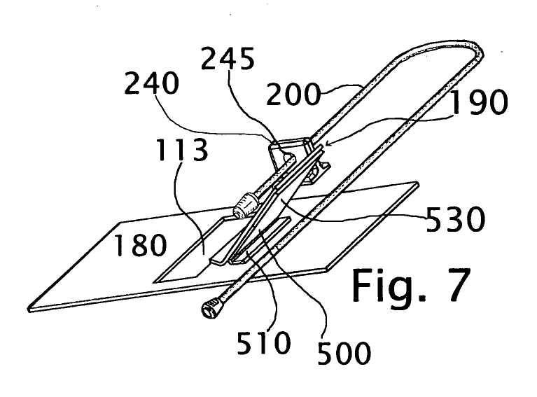

FIG. 7 is the invention with a medical tube threaded through a medical tube anchor view from the film layer side.

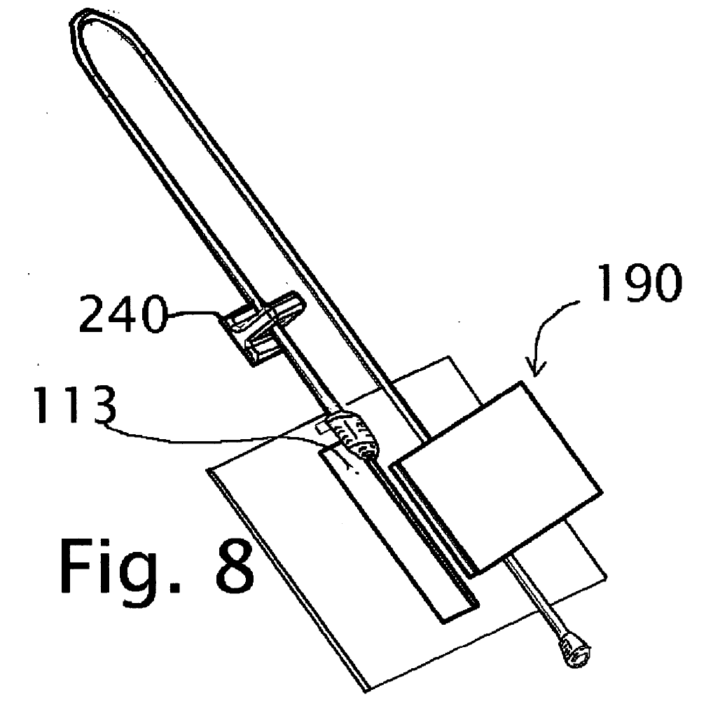

FIG. 8 the embodiment of FIG. 7, viewed from the deployer side.

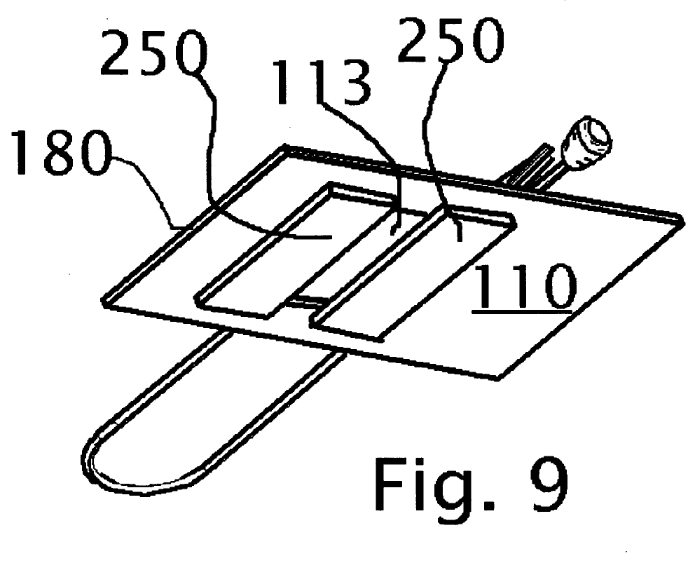

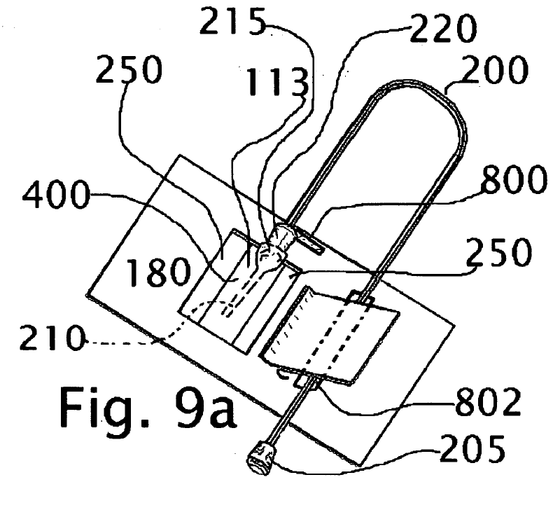

FIG. 9 is a bottom view of the dressing with 2 absorbent pads affixed to the bottom of the bandage base.

FIG. 9a is a view of the dressing of FIG. 9, from the top side.

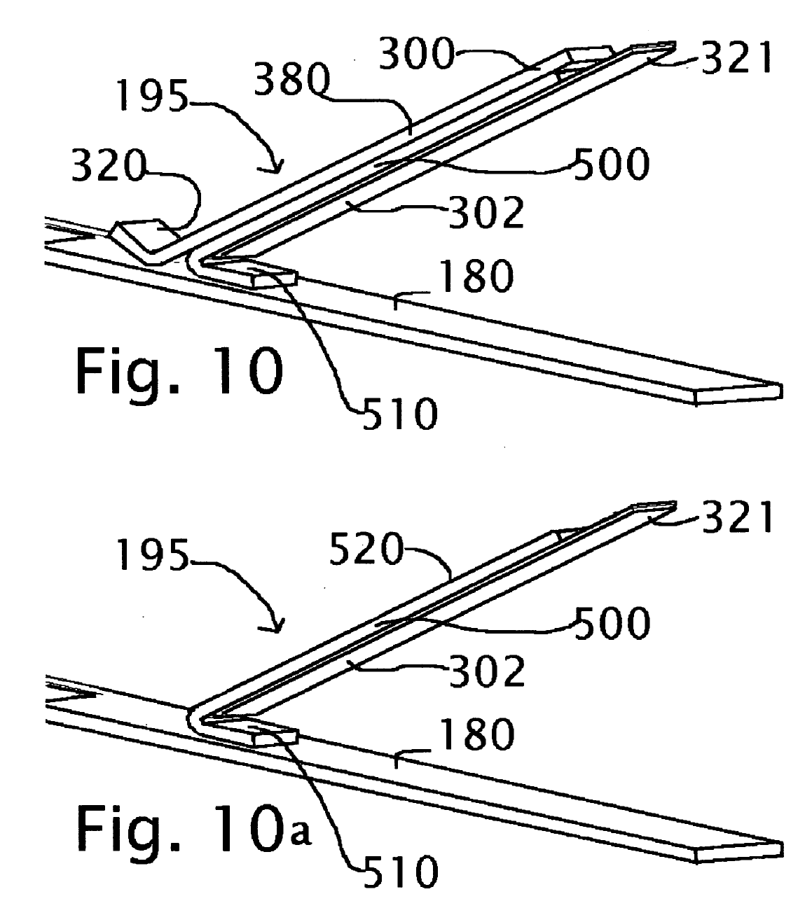

FIG. 10 is a close-up of the invention disclosing an alternate 3-layer deployer door.

FIG. 10a is the embodiment of FIG. 10, with the top deployer removed.

FIG. 11 is an oblique exploded view of a two-element embodiment of the invention.

FIG. 11a is an assembled view of the embodiment of FIG. 11.

FIG. 12 is an oblique view of a re-positionable bi-digital tube anchor.

FIG. 12a is a frontal view of a tunnel version tube anchor.

FIG. 12b is an oblique view of a tube anchor with flanges anchoring an I.V. tube.

FIG. 12c is the tube anchor of FIG. 12b without the I.V. tube.

FIG. 13 is an oblique view of a 2-piece embodiment of the invention.

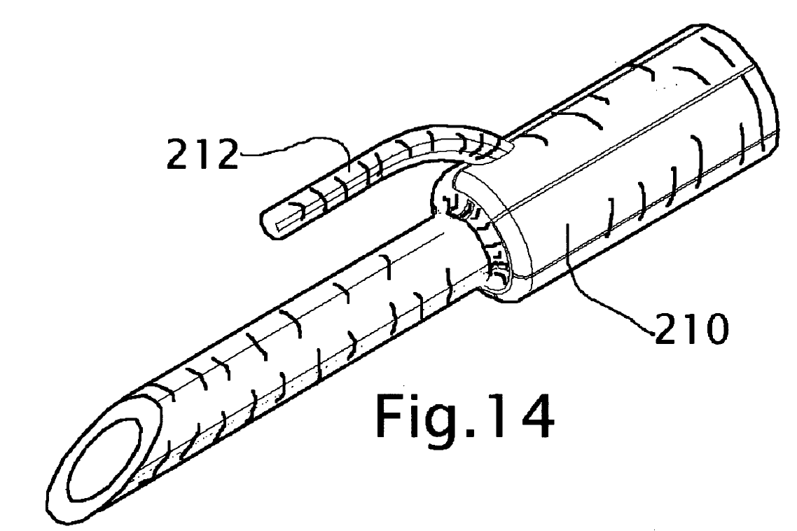

FIG. 14 is an oblique view of an I.V. catheter with a grabber extension.

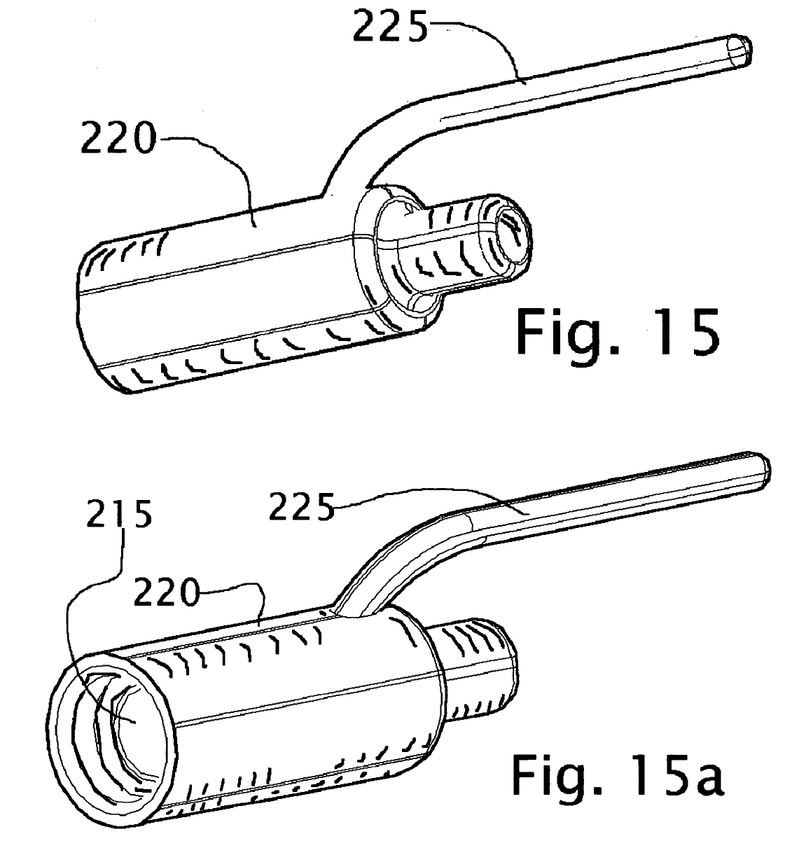

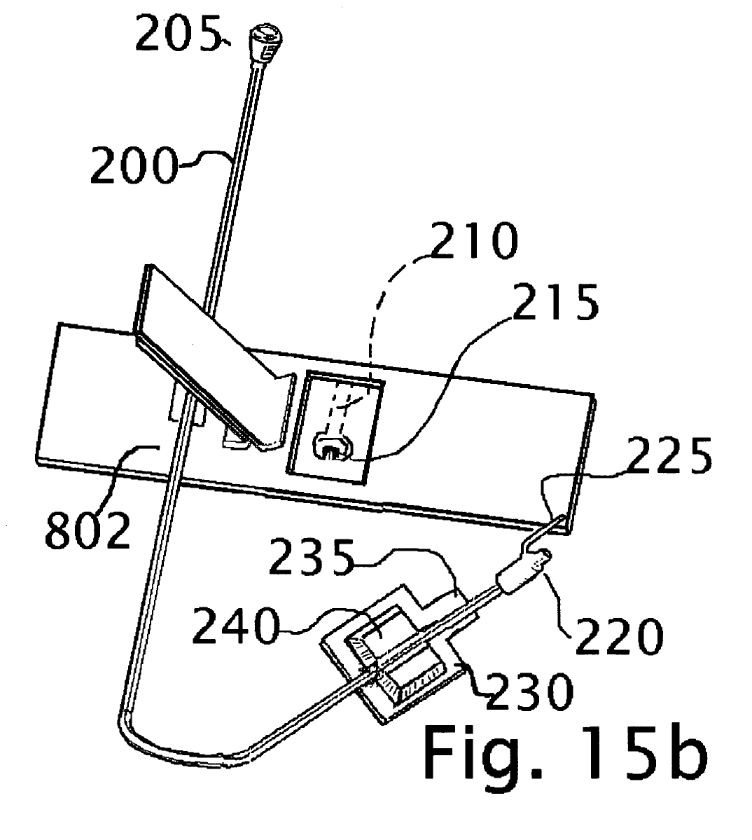

FIG. 15 is an oblique view of an I.V. catheter adapter with a grabber extension from the male end.

FIG. 15a is an oblique view of the I.V. catheter adapter of FIG. 15 from the female end.

FIG. 15b is the embodiment of FIG. 5, with the grabber extension I.V. catheter adapter with extension mounted on the I.V. tube extension.

### DETAILED DESCRIPTION OF THE INVENTION

Before discussing the structure of the invention in detail, note that the layers of materials used in the structure are quite thin. In the various figures, the thicknesses are sometimes exaggerated for clarity of illustration. In particular, layers of adhesive are usually not shown, as the adhesive is generally coated directly onto the components and is not technically a separate part. All adhesive coated parts are clearly identified and the sides and portions of the components that are adhesive coated are clearly defined. Recognize, also, that when exaggerations occur, they also exaggerate the curvatures that occur in the drawings at the overlapping intersections of various layers.

The present invention, as seen in FIG. 1. is a preemptive dressing 700 for use with invasive medical procedures, typically for use with procedures wherein a catheter or a needle is placed in the lumen of a blood vessel. This device shown 700 is configured to be placed on the skin of a patient, prior to introducing a medical device into the lumen of said vessel. The dressing 700 is placed on skin so that the blood vessel to be catheterized is visible and accessible via an aperture 113 in a bandage base 180. A deployment door 190 is shown, which is the aggregate of a deployer 380 and a bandaging film layer 500. The deployer has two sections, the deployer body 325 and a retention extension 320 which is further separated into two separate extension legs 360 362.

The invention 700 is shown in FIG. 2 with the deployer 380 FIG. 1 absent. In this view, it can be seen that the film or bandaging film material 500 is hingedly attached to non-adhesively coated side 118 of bandage base 180 by hinge 560 which is a flap the adhesive layer 520 of film material 500 folded under and affixed to said bandage base 180. The hinge 560 in film 500 is approximately parallel to the transverse edges of aperture 113 in bandage base 180. The aperture 113 herein is rectangular, but nothing in this disclosure is intended to limit the size, shape, or location of said aperture 113. FIG. 3 is a similar embodiment to the embodiment of FIG. 1. It differs in that there is only one retention extension 320, which functions as a pull-tab of deployer 380 and bandage base 180 flaps 114 115, are long. The long flaps 114 115 are so the bandage base 180 of the device can be wrapped around a limb and adhered to itself, to make it more secure on the patient.

In veterinary medicine, catheter securement has challenges beyond those of human medicine. Animals typically have an abundance of hair or fur and often must be restrained during the catheterization procedure. Therefore, a method of secure and rapid catheter stabilization would be valuable. The embodiment of FIG. 4 is the same as that of FIG. 3. FIG. 4 shows the two flaps 114 115 of bandage base 180 wrapped around a limb, and adhered together by an adhesive clasp 117. The skin 400 of a limb 450, and a sub-cutaneous blood vessel 410 are visible through aperture 113.

FIG. 5 is an embodiment of the invention that includes two separate methods of stabilizing the medical line. It also shows a medical line extension 200 that can be affixed to the dressing by adhesive surface 802 to preemptively stabilize the line and further facilitate the catheterization process. When said extension 200 is used, the medical line coming from a fluid supply can be changed at the I.V. tube extension hub receiver 205 rather than at the procedure site. Using this format, the provider is further away from the catheter 210 and the I.V. tube male catheter adapter 220, therefore there is less chance of exposure to bloodborne pathogens when changing I.V. lines. This embodiment also shows an island 230 and an island tongue 235, composed of bandage base material and island 230 and island tongue 235 have a carrier paper layer on the bottom. The adhesive surface of tongue 235 can be exposed by removing carrier paper, not shown, from the bottom. Then tongue 235 can be adhered to bandage base 180. An I.V. line anchor 240 is adhered to island 230. The I.V. line 200 can be retained by retainers such as an I.V. line anchors of any common commercially available type, such as those manufactured by Venetec International Inc, San Diego, Calif. or by the I.V. line anchors disclosed in this application. Tongue 235 allows the I.V. line extension 200 and the male hub 220 on the I.V. line extension 200 to be secured, yet still to be moved small distances, such as far enough for hub adapter 205 to be engaged to and disengaged from female catheter hub 215.

FIG. 5a is exactly like FIG. 5, except that bandage base flap 115 is shortened. This embodiment shows that a large bandage base flap 115 with or without aperture 113 is not required. This embodiment is simpler and less expensive to manufacture than any of the others disclosed here in the previous drawings. The embodiment of FIGS. 11 and 11a are even less expensive to manufacture than embodiments of FIG. 5a.

FIG. 6 shows an important feature, the film anchor-flap 510 which is a folded edge of film 500 that anchors the deployment door 190 to the bandage base 180. FIG. 6 is the same as FIG. 4 except that an I.V. line extension 200 is shown adhered to the bandage base 180 near the film anchor-flap 510 of film 500 by an unseen adhesive. This drawing demonstrates one method of pre-stabilizing the I.V. line, prior to inserting the catheter. A film anchor-flap 510 is present on every embodiment.

FIG. 7 shows an embodiment of the invention 700 wherein the deployment door 190 is narrower than the aperture. When this embodiment is deployed, a catheter, not shown, will be covered and stabilized by a band, rather than a complete layer, of bandaging film 500. In this view, the deployment door 190 is viewed from the back, the non-adhesively coated, side 530 of film layer 500. A medical extension tube 200 is threaded through a tube anchor 240. Medical tube anchor 240 has an adhesive coating on the bottom surface so that before or after the device 700 is deployed, the tube can be anchored to skin or to an island 230. The coating on the bottom of tube anchor 240 cannot be seen, but FIG. 12a, which shows anchor 240 in more detail and discloses a carrier paper layer on the bottom, protecting the unseen adhesive surface. The film anchor-flap 510 can be well visualized in this view.

FIG. 8 is an embodiment that includes a tube anchor 240 that comprises an aperture or tunnel 245 with a section of a medical tube extension threaded 200 through it. This is the embodiment of FIG. 7, viewed from the front.

FIG. 9 is a bottom view of the dressing with two absorbent pads 250 affixed to the adhesive coated bottom 110 of bandage base 180. The plurality and the locations of the absorbent pads are arbitrary and non-limiting. The invention may have no absorbent pads or one or a plurality of pads 250 and the absorbent pads may be adhered to the film layer 500 or they may be adhered to the bandage base layer 180 and they may vary in size dramatically.

FIG. 9a is a view of the dressing of FIG. 9, from the top side. This view shows locations where adhesive coatings, adhesive pads, or double sided tape can be placed 800 802 to use as tube anchor pads. In this view, a catheter adapter 220 is weakly affixed to the bandage base 180 by a small rectangle of adhesive 800 and an I.V. line 200 is anchored to bandage base 180 by an adhesive member 802. These adhesive members 800 802 are non-limiting examples of I.V. line anchoring means. A catheter 210 is seen in phantom because it is inserted under skin 400. A male catheter adapter 220 is plugged into catheter female hub 215 and I.V. extension line 200 has a female luer adapter 205 on the receiving end. 802

FIG. 10 is a close-up of the invention disclosing an alternate embodiment of the deployment door, the three-layer deployment door 195. Three-layer deployment door 195 features a medical bandaging film 500 sandwiched in between two carrier paper layers 300 302. The carrier papers 300 302 may be similar or they may be of very different varieties of carrier paper, wherein one is a heavy card thickness coated paper and the other is no more than a coated-on stiffening layer that can be peeled off during deployment.

FIG. 10a: This is the embodiment of FIG. 10 with the top deployer 380 removed. This embodiment is used by removing deployer 380 FIG. 10, thereby exposing the adhesive surface 520 of film 500, the placing a catheter in the vessel, and then closing door 195, now only two-layers, over the procedure site. This blankets the catheter and the bandage base with the adhesive surface 520 of film 500. This immediately affixes the film to the catheter and to the dressing and stabilizes the catheter with stiffening layer 302 on top and extension 321 positioned so it can be grasped and pulled off of the dressing, leaving film 500 covering the catheter and the aperture.

The basis for this invention is a specialization of the inventor's previous invention disclosed in application the inventor's pending utility application Ser. No. 10/906,630. The invention of that application is a preemptive invasive site dressing similar to that of FIGS. 11 and 11A. The 10/90660 dressing most commonly comprised an absorbent pad and this one most commonly does not. 10/906,630 does not speak to catheter anchoring, which is the essence of the present invention. FIGS. 11 and 11A a show an embodiment of the present invention that comprises a deployment door 190. While the invention could not easily be packaged in this configuration without the addition of a carrier paper layer on the adhesive surface 520 of the film anchor-flap 510, this embodiment does, in fact, speak to the heart of the invention and function as desired to preemptively secure a catheter. The embodiment of FIGS. 11 and 11a are fully functional for the purpose of preemptively dressing an injection site or a catheterization site. The deployment door 190 comprises a bandaging film layer 500, and a deployer 380. The deployer further comprises a deployer body 325 and two retention legs 360 362 and the film layer 500 further comprises a film anchor-flap 510 and a flip-door 550. The layer of bandaging film 500 is adhered to deployer 380 by adhesive surface 520 of said bandaging film 500. A film anchor-flap 510 is located between the two retention legs 360 362. When the deployment door 190 is placed on the catheterization site and folded back or cocked, as disclosed in 10/906630, it can be deployed over the catheter site to secure a catheter or over an injection site to cover the injection puncture.

Since bandaging films of these type can be very thin and difficult to handle and because they tend to fold back upon themselves and stick to themselves is a way that makes their utility unrecoverable, a deployment door embodiment, similar to that of FIGS. 11 and 11a, is foreseen, comprising a 3-layer deployment door 195, like that of FIG. 10. Three-layer films of this type are available commercially from 3M and Avery Label, Medical Tape Division.

FIGS. 12 through 12d are examples of tube anchors that can be used in conjunction with the dressing. FIG. 12 shows an anchor 240 that has an expansion retention ring or a medical tubing expansion retention anchor clamp 242 with a tension release tab or clamp handle 243. An I.V. tube 200 is shown in cross section 202 threaded through the expansion retention ring or clamp 242. The anchor 240 has a base 241 with an adhesive coated bottom—not visible. The adhesive coated bottom has a layer of release paper 304 releasably affixed to the bottom surface and release paper 304 has a pull-tab 306 to remove release paper 304 from the bottom of anchor 240.

FIG. 12a is the same as FIG. 12 except that in 12a, instead of the medical tubing expansion retention anchor clamp 242 shown in FIG. 12, this anchor 240 has a solid body 241 with a retention tunnel 245 though the body of tube anchor 240. To use this anchor 240 FIG. 12a, a tube 200 is threaded through tunnel 245 before catheter adapters are molded to the tube. FIGS. 7 and 8 each show a tube 200 threaded through a tunnel of a tube anchor of this embodiment. FIGS. 12, 12a, and 12b show I.V. tube 200 in cross-section 202.

FIG. 12b is a tube anchor 240 that uses a plurality of flexible fingers to hold the I.V. tube in place. The flexible fingers or flanges 247 are visible in FIG. 12c and are easier to see because the tube 200 in the tunnel 245 has been removed.

FIG. 13 is a very elementary embodiment of the dressing 700 comprising only a film bandage base layer 500 and a piece of carrier paper 300. The film bandage base layer 500 further comprises an anchor-flap 510 and an adhesive coating 520 on the underside of film 500 and a hinge 560. Carrier paper layer 300 further comprises a deployer body 325 and a retention extension 320. The deployer body 325 is part of the main body of carrier paper 300 up to where it bends and becomes retention extension 320.

FIG. 14 is a modified I.V. catheter 210 with a modification. Catheter 210 has a small flexible grabber extension 212 that parallels the needle. Said extension 212 remains outside the skin when the catheter is placed in the lumen of a blood vessel. The purpose of the extension is to extend the length of the extra-cutaneous physical presence of the catheter after the catheter has been placed. This results in bandaging film 500 having a larger target to grab when the dressing is deployed.

FIG. 15 is a catheter adapter 220 with a catheter adapter grabber extension 225.

FIG. 15a is the male catheter adapter 220 of FIG. 15 from a different angle so that the back or female hub receiver 215 is viewable. The relative length of the grabber extension is non-limiting. Grabber extensions which are larger or smaller or longer or shorter would still fall within the scope of the invention.

FIG. 15b is the catheter adapter 220 of FIGS. 15 and 15a on the I.V. tube extension of the embodiment of FIG. 5.

1. An I.V. catheter anchoring dressing comprising a deployment door, which further comprises a bandage base film layer; wherein said bandage base film layer comprises an adhesive coating on a bottom face; and a carrier paper layer; wherein said carrier paper layer is composed of material designed to affix to and release from said adhesive layer; and said carrier paper layer is releasably affixed to a predetermined part of the adhesive layer of said bandage base film layer; and a predetermined part of said adhesive coating is exposed, such that said dressing can be hingedly affixed to a surface; and said dressing further comprises a means whereby said dressing can be cocked.

2. The I.V. catheter anchoring dressing of claim 1, whereby said dressing further comprises a bandage base layer; wherein said bandage base layer comprises an adhesive coating on a bottom face of said bandage base; and said I.V. catheter anchoring dressing of claim 1 is hingedly affixed to said bandage base layer.

3. The I.V. catheter anchoring dressing of claim 2, wherein said dressing further comprises means for limiting the movement of an I.V. line.

4. The I.V. catheter anchoring dressing of claim 1, wherein said deployer comprises at least one retention extension.

5. The I.V. catheter anchoring dressing of claim 3, wherein said means for limiting the movement of an I.V. line comprises at least one adhesive tape member, whereby affixing said I.V. line to said adhesive tape member, limits spontaneous movement of said I.V. line.

6. The I.V. catheter anchoring dressing of claim 3, wherein said means comprises a retainer, wherein said retainer is configured to receive and retain an I.V. line tube.

7. The I.V. catheter anchoring dressing of claim 3, wherein said dressing further comprises a flexible and positionable island and said island comprises bandage base material; and said island comprises a means whereby said island can be affixed to skin, and said island further comprises a means whereby said island can be affixed to said dressing; and one surface of said island is exposed, such that a means to limit the movement of an I.V. line can be adhered to said island.

8. The IV catheter anchoring dressing of claim 6, wherein said island further comprises a means to limit the movement of an I.V. line.

9. The IV catheter anchoring dressing of claim 6, wherein said island comprises a surface, whereby a means to limit the movement of an I.V. line can be affixed.

10. The I.V. catheter anchoring dressing of claim 2, wherein said bandage base further comprises a through and through aperture, through which a predetermined section of the tissue of said patient can be accessed to perform an invasive medical procedure.

11. The I.V. catheter anchoring dressing of claim 10, wherein the said bandage base is of adequate width to wrap said bandage base fully around the limb of a patient and the ends of said bandage base comprise a means to affix one to the other, such that said dressing is further stabilized and movement of said dressing is limited.

12. The I.V. catheter anchoring dressing of claim 1, wherein said film layer further comprises a removable stiffening layer, whereby the shape of said film layer is maintained by a stiffening means and said film can be easily moved, and, when moved, said stiffening layer inhibits said film layer from folding or distorting and said adhesive surface of said film layer is inhibited from contacting itself and whereby said adhesive surface of said film is prevented from affixing to itself.

13. The I.V. catheter anchoring dressing of claim 8, wherein said I.V. tube extension further comprises an I.V. tube anchor whereby said tube is threaded through an aperture in said tube anchor, and said tube anchor has an adhesive layer on a bottom surface, such that said tube anchor can be adhered to skin, whereby said tube anchor will limit the movement of said I.V. tube such that said I.V. tube can be moved only towards and away from said medical procedure site perpendicular to a transverse cross section of said dressing, and said tube can be released and it will remain more or less where it was when it was released.

14. The I.V. catheter anchoring dressing of claim 2, wherein said bandage base layer further comprises an absorbent layer and said absorbent layer is smaller than said bandage base layer, and said absorbent layer comprises one or more absorbent pads, wherein said absorbent pads may vary in thickness from less than 1/32″ to greater than 1″.

15. The I.V. catheter anchoring dressing of claim 1, wherein said dressing further comprises an I.V. catheter adapter, which further comprises a catheter adapter grabber extension.

16. The I.V. catheter anchoring dressing of claim 1, wherein said dressing further comprises an I.V. catheter, which further comprises a catheter adapter grabber extension.

17. A method of stabilizing a vascular catheter comprising the steps of; preparing skin for an invasive procedure; and adhering a dressing near the vessel to be catheterized; and puncturing skin and a blood vessel with a vascular catheter, and placing said catheter into the lumen of said vessel; and pulling a deployer door across the operative field; while peeling said deployer off of the adhesively coated surface of a bandaging film; thereby bringing said adhesively coated surface of said film into contact with said catheter; and smoothing said film against said catheter; thereby setting said adhesive to said catheter; and pushing said film against said catheter and said skin; whereby said catheter is securely affixed and secured between said skin and to said dressing.

18. An I.V. catheter hub adapter wherein said hub adapter further comprises a catheter grabber extension.

19. The I.V. catheter hub adapter of claim 18 wherein said catheter grabber comprises a flexible material.

20. The I.V. catheter hub adapter of claim 18 wherein said catheter hub adapter is fully integrated into the hub of an I.V. catheter.
